(19) United States
(12) Patent Application Publication
(10) Pub. No.: US 2010/0324457 A1
(43) Pub. Date: Dec. 23, 2010

(54) SKELETAL-MUSCULAR POSITION MONITORING DEVICE

(75) Inventors: Jacob Bean, Shelley, ID (US); Bradley James Hollingworth, Irving, TX (US); Nathan Odell Seaver, Longmont, CO (US); Allen Bruce Gilbert, Rexburg, ID (US); Jeffrey Jess Lynn, Rexburg, ID (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Jacob Bean, Shelley, ID (US)

(21) Appl. No.: 12/635,468

(22) Filed: Dec. 10, 2009

Related U.S. Application Data
(60) Provisional application No. 61/121,361, filed on Dec. 10, 2008.

(51) Int. Cl. A61B 5/103

(52) U.S. Cl. 600/595

(57) ABSTRACT

A system is disclosed that records position data for portions of a body as a function of time. The position data can be collected from one or more sensors secured to the body either individually or using a patch. The sensors, in some embodiments, can include stretch sensors that produce a change in electrical resistance as the stretch sensors are stretched. A data logger can be used to record the data. Various other elements such as a feedback mechanism or a manual pain indicator can also be included.

### CROSS-REFERENCES TO RELATED APPLICATIONS

This patent application is a non-provisional claiming priority benefit of U.S. provisional patent application Ser. No. 61/121,361, filed on Dec. 10, 2008 and entitled “Back Position Monitoring Device,” the entire disclosure of which is herein incorporated by reference.

### BRIEF SUMMARY OF THE INVENTION

Embodiments of the present invention include skeletal-muscular position and/or motion sensing devices. Such devices can be used by individuals undergoing therapy, habit breaking, posture improvement, athletic training, etc. In some embodiments of the invention, devices can include sensors that can be attached to a body at various positions in order to measure the position and/or movement of a specific portion of the body. The sensors can include stretch sensors and/or sensors that return the distance between adjacent sensors. The sensors can also include one or more tilt sensors. A data logger can be used to measure and record electrical signals that can be correlated with the sensor stretch, position, and/or tilt. The data logger can record sensor data periodically or continuously.

In some embodiments of the invention, a pain indication button can be used by a user to indicate pain resulting from a position or movement. The data logger can record data indicating a painful situation has occurred.

In some embodiments of the invention, one or more sensors can be coupled with a patch. The sensors can be prearranged according to various sensing configurations.

### BRIEF DESCRIPTION OF THE DRAWINGS

FIG. 1 shows a skeletal-muscular position mentioning device secured on the lower back of an individual in one configuration.

FIG. 2 shows a skeletal-muscular position mentioning device secured on the lower back of an individual in another configuration.

FIG. 3 shows a skeletal-muscular position mentioning device secured on the lower back of an individual in yet another configuration.

FIG. 4 shows a skeletal-muscular position mentioning device secured on the lower back of an individual in another configuration.

FIG. 5 shows a skeletal-muscular position mentioning device secured on the lower back of an individual in yet another configuration.

FIG. 6 shows a skeletal-muscular position monitoring device placed on the leg of an individual in one configuration and an skeletal-muscular position monitoring device placed on the arm of individual in one configuration.

FIG. 7 shows a skeletal-muscular position monitoring device placed on the leg of an individual in one configuration and an skeletal-muscular position monitoring device placed on the arm of individual in one configuration.

FIG. 8 shows a skeletal-muscular position monitoring device secured on the neck of an individual in one configuration.

FIG. 9 shows another skeletal-muscular position monitoring device secured on the neck of an individual in one configuration.

FIG. 10 shows a skeletal-muscular position mentioning device secured on an adhesive patch according to one embodiment of the invention.

FIG. 11 shows a block diagram of a computational device that can be used to collect sensor input according to some embodiments.

FIG. 12 shows a flowchart of a process that can be used to record body position data and provide sensory feedback according to some embodiments.

### DETAILED DESCRIPTION OF THE INVENTION

Many people suffer from back injury, recurring back pain, neck pain, or pain in their arms or legs. While current medical research is expanding the understanding of the problem of back pain, relatively little information is available on dynamic and static positions and mechanics of the skeletal-muscular position as a function of time. Often information obtained from a patient seeking medical treatment is unclear about the cause of pain or trauma. Patients often find it difficult to describe what positions or activities precede an episode of pain or other sensations associated with musculoskeletal dysfunction. Assessing the posture and activities of patients with skeletal-muscular injuries is an essential part of clinical evaluation and treatment.

In some embodiments of the invention, skeletal-muscular movement and/or position data can be recorded by various sensors including tilt sensors and/or stretch sensors. This data can be used to establish baseline mechanics and/or responses to treatments such as exercise and postural training. In other embodiments, the data can be used by a data logger to provide instantaneous feedback to the user when their body is placed in a potentially painful position or when their body is in an posturally inferior position. One embodiment of the present invention provides a wearable device that records normal movement of the back or other body parts over time. The device can be worn such that the user can wear the device without altering their normal movements and/or clothing. While wearing the device an individual can attend to normal daily activities. The device can also be designed to be worn for long periods of time and can continuously monitor position data.

In some embodiments of the invention baseline values can be recorded when the device is initially attached with the patient. These baseline values can be used to calibrate the device and/or to reposition sensors. For example, when one or more sensors have been attached with a patient, the patient can be requested to position themselves in a neutral position to determine a baseline neutral position. The patient can then be asked to flex and/or contract a muscle and/or move or rotate a limb, their back, and/or neck to an extreme position for baseline positions. Such extreme positions can include full range of available movements for a given part of the body such as flexion, extension, rotation, lateral flexion, etc.

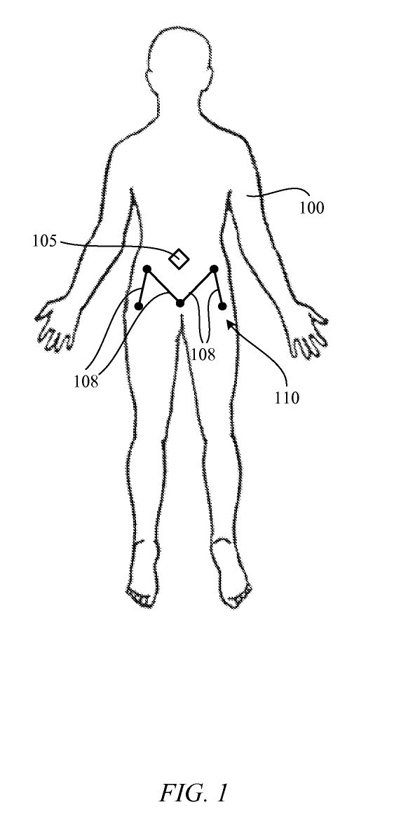

A skeletal-muscular position mentioning device, for example, can be used to monitor the position and/or movement of the back. For instance, FIG. 1 shows a back position monitoring device placed on the lower back of an individual in M-shaped configuration 110. M-shaped configuration 110 can include four stretch sensors. On each side of the back stretch sensor 108 can start at the illiac crest and extend up to the lateral aspect of the lower ribs. Two more stretch sensors 108 can extend from the lateral aspect of the lower ribs on each side of the body down to the superior sacrum on the lower back of an individual.

Tilt sensor 105 can also be secured to the back of individual. Tilt sensor 105 can measure the angle of the tilt sensor relative to the Earth surface and/or relative to the gravitational vector. Tilt sensor 105, for example, can include an inclinometer or an accelerometer. Tilt sensor 105 can be used to determine the individual's position in relation to gravity. Tilt sensor 105 can be placed at any number of positions on the body. Tilt sensor 105 can include a two or three axis tilt sensor.

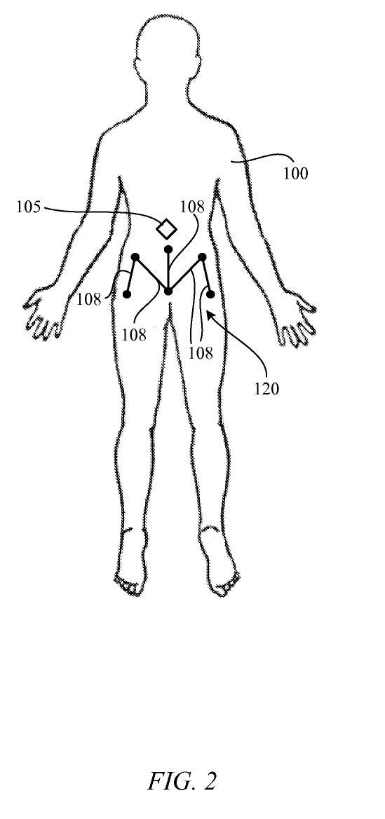

The number of configurations of sensors positioned on a body are nearly endless. Sensors can be positioned to measure any skeletal and/or muscular activity of the body and can be arranged accordingly. FIGS. 2-9 show some examples of sensor configurations.

FIG. 2 shows a skeletal-muscular position mentioning device secured to the lower back of an individual in another configuration. This configuration 120 is similar to the M-shaped configuration with an additional stretch sensor 108 extending along the back bone of the individual. Tilt sensor 105 can also be coupled with the individual at any position.

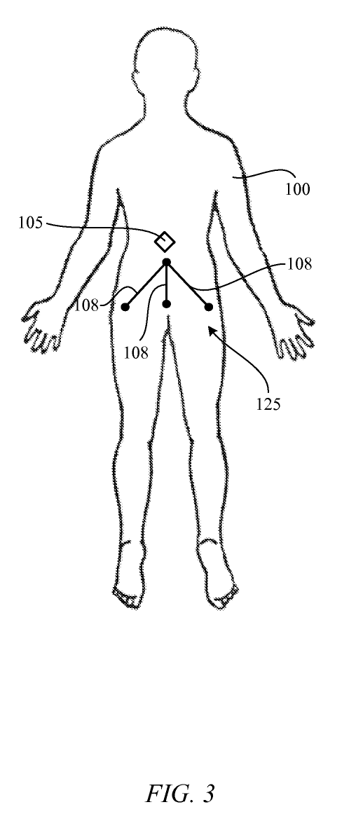

FIG. 3 shows another skeletal-muscular position mentioning device secured to the lower back of an individual in one configuration. In this configuration 125 three sensors are used. One stretch sensor 108 on each side of the body can start at the illiac crest and extend up to a position on the backbone near the lower back. Another stretch sensor 108 can extend along the backbone starting at the superior sacrum up to the same point on the backbone. Tilt sensor 105 can also be coupled with the individual at any position.

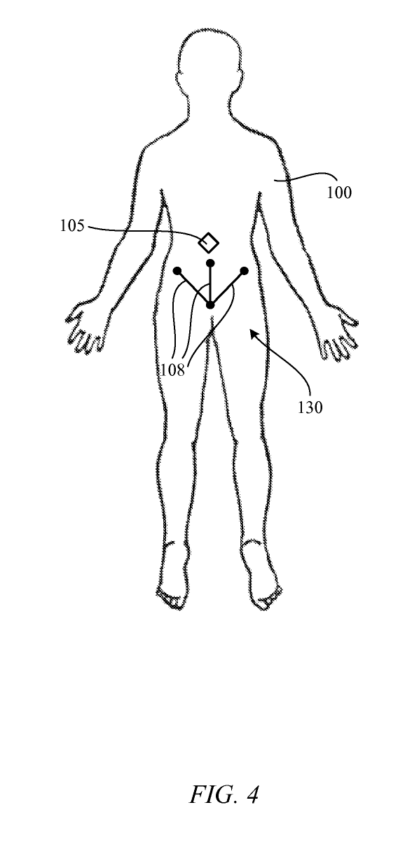

FIG. 4 shows a skeletal-muscular position mentioning device secured to the lower back of an individual in another configuration. In this configuration 130 three sensors are also used. Stretch sensors can extend from the lateral aspect of the lower ribs down to the superior sacrum on the lower back on each side of the body. Another stretch sensor 108 can extend along the backbone starting at the superior sacrum. Tilt sensor 105 can also be coupled with the individual at any position.

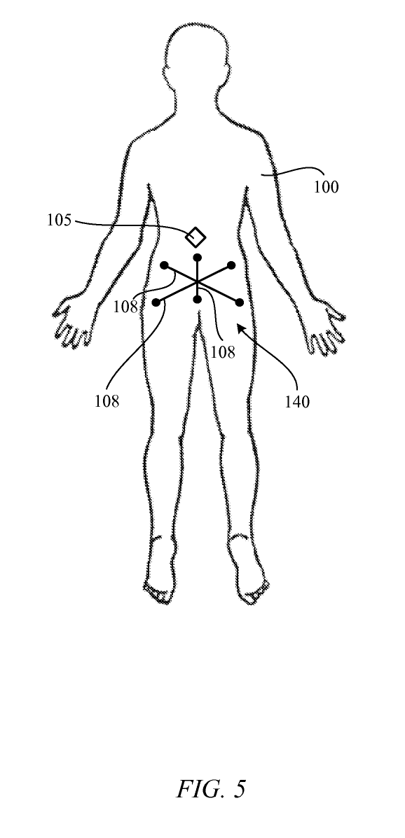

FIG. 5 shows a skeletal-muscular position mentioning device secured to the lower back of an individual in another configuration. In this configuration 140 three sensors are used. One stretch sensor 108 can extend along the backbone starting at the superior sacrum. Two other stretch sensors can extend across the back between the illiac crest and extend up to a position across the body to the lateral aspect of the lower ribs on the opposite side of the back as shown. Tilt sensor 105 can also be coupled with the individual at any position.

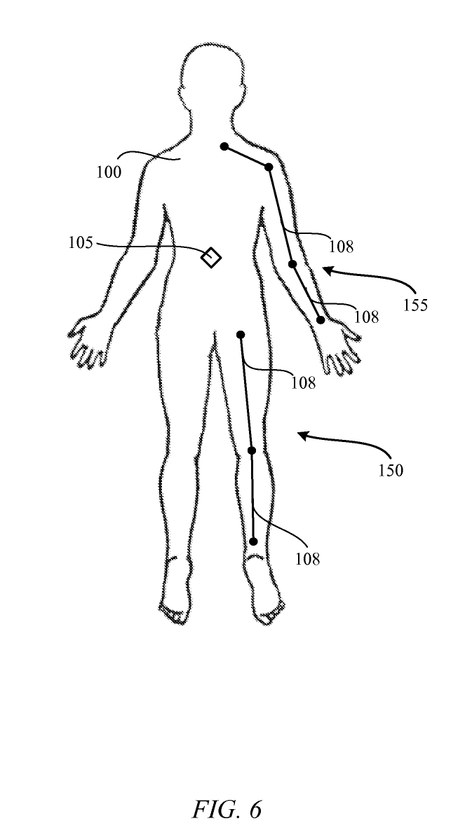

FIG. 6 shows a skeletal-muscular monitoring device placed on the leg of an individual in one configuration 150. In this configuration two stretch sensors 108 are placed across the knee with an anchor position at the joint. In other embodiments stretch sensors can also be placed across the hip and/or ankle with an anchor position at the joint. FIG. 6 also shows a skeletal-muscular monitoring device placed on the arm of an individual in one configuration 155. In this configuration two stretch sensors 108 are placed across the elbow with an anchor position at the joint. In other embodiments stretch sensors can also be placed across the shoulder and/or wrist with an anchor position at the joint. Tilt sensor 105 can also be coupled with the individual at any position.

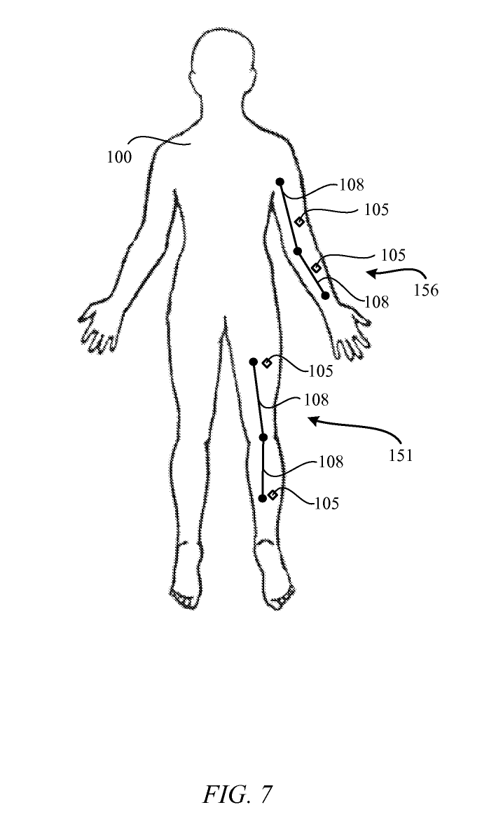

In some embodiments multiple tilt sensors 105 can be used instead of or in conjunction with a tilt sensor placed on the back of a user. Any number of tilt sensors can be used. FIG. 7 shows skeletal-muscular monitoring devices in configuration 151 and 156. In both configurations tilt sensors are coupled to different portions of the limbs. Various other configurations can be used. Tilt sensors 105 can be used to show rotation and other movement of body parts in relation to other body parts.

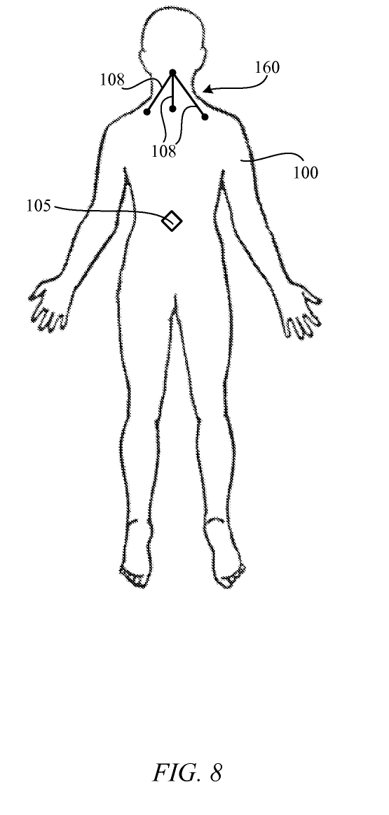

FIG. 8 shows a skeletal-muscular position monitoring device 160 secured to the back of the neck of individual 100 in one configuration. Three stretch sensors 108 are shown. One extends along the upper back bone. The other two extend from top of the back bone laterally toward the shoulders. In this configuration the sensors can measure the relative movement of the neck. Tilt sensor 105 is also secured to the body.

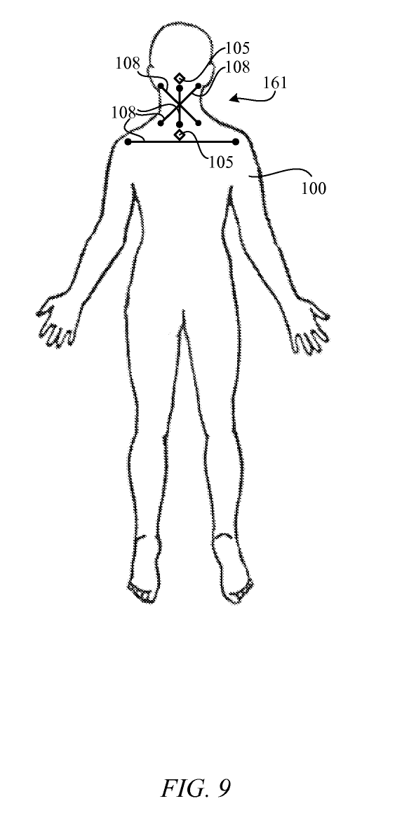

FIG. 9 shows a skeletal-muscular position monitoring device 161 also secured to the back of the neck of individual 100 in another configuration. Four stretch sensors 108 are shown. One stretch sensor 108 can be placed longitudinally with the caudal anchor on the skin at the spinous process of the 7^{th} cervical vertebrae and the cephalic anchor at the level of the 4^{th} cervical vertebrae. Second stretch sensor 108 can be anchored at the 4^{th} cervical vertebrae inferiorly and superiorly anchored at the occiput. Additional stretch sensors 108 can be anchored lateral to the occiput superiorly and descend medially and inferiorly to an anchor at the area of the 4^{th} cervical vertebrae. Two additional stretch sensors 108 are anchored superiorly above the 4^{th} cervical vertebrae and descend laterally and inferiorly to anchor on the lateral base of the neck midway between the spine and the shoulder joint. Final stretch sensor 108 can be anchored over the spine of each scapula. Two tilt sensors 105 are positioned at the spinous process of the 7^{th} cervical vertebrae and the occiput.

In some embodiments, stretch sensor 108 can change resistance when stretched. For example, when relaxed the sensor material has a nominal resistance, of 1000 ohms per linear inch. As stretch sensor 108 is stretched the resistance can gradually increase. When the sensor is stretched 50% its resistance can approximately double to 2.0 Kohms per inch. Various other stretch sensors can be used with various values of resistance and changes in resistance per stretch. In some embodiments, the sensor can be a flexible cylindrical cord 0.060-0.070 in diameter, with spade or ring electrical terminals at each end. Stretch sensors 108 can be any length necessary depending on the application and/or patient.

In some embodiments a stretch sensor can be enclosed within a protective device such as a tube, casing, straw, sheath. A protective device can protect the stretch sensor from pinching or other damage. For example, a stretch sensor disposed within a protective device can stretch within the protective device.

Various other sensor types can be used. In some embodiments, sensors can be used to convert skeletal and/or muscular movement of one part of the body relative to some other point on the body into some type of electrical property (e.g., resistance, voltage, current, etc.). Moreover, sensors can be arranged in any configuration. Some sensors can be placed at specific joints to record the rotation and/or motion of a joint. Other sensors, such as an electromyographical sensor, may also be used to detect muscle activation in conjunction with positional changes. Sensors placed over certain muscle groups can detect the electrical properties of a muscle when the muscle is contracting.

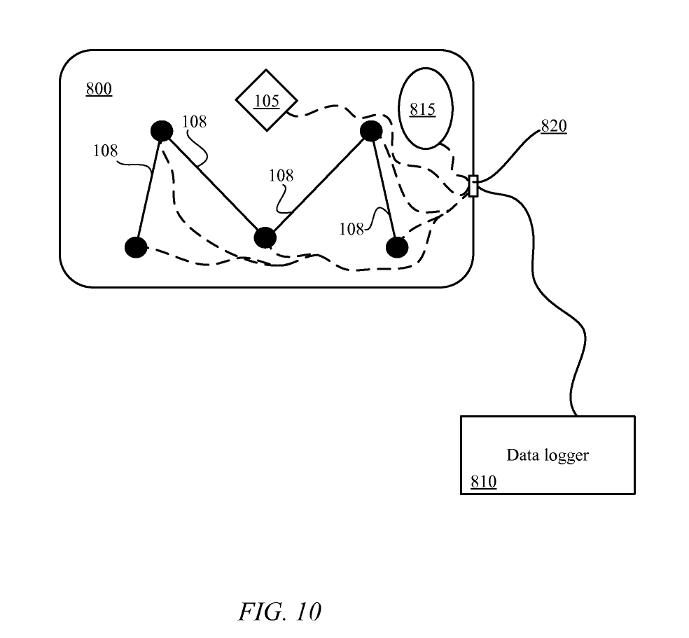

Sensors can be placed on the body using various techniques. One such technique includes using a patch or adhesive bandage with one or more sensors previously coupled with the patch. A user can then simply attach the patch to their body. FIG. 10 shows a patch with stretch sensors 108 arranged in the M-configuration as shown in FIG. 1 coupled with patch 800, which can be any type of adhesive or bandage. In some embodiments, patch 800 can also include tilt sensor 105. In other embodiments, tilt sensor 105 can be coupled with another or different patch than the patch coupled with the stretch sensors. Patches can vary in size and/or in configuration.

Connector 820 can be used to electrically connect stretch sensors 108 with data logger 810. Wires can interconnect stretch sensors 108 and/or tilt sensor 105 with connector 820. In some embodiments, connector 820 can be a low resistance connector. In other embodiments, connector 820 can have a known but stable resistance. Connector 820 can be coupled with a wire bus that can be coupled with data logger 810. In some embodiments of the invention, the sensors can be coupled with the data logger without a connector on the patch.

Patch 800 can also include feedback device 815. Feedback device 815 can include a vibrator or a shocker. Feedback device 815 can provide physical feedback to a user when the data logger 810 determines that the user's body is in a position that may be potentially painful. For example, feedback device 815 can provide feedback by vibrating or with an electrical shock. In some embodiments, feedback can be provided not only in cases of pain, but also for training purposes for a user to correct for bad posture or another physical habit.

While stretch sensors in the M-configuration are shown coupled with a patch in FIG. 10, any type of sensor can be used in any configuration. Multiple patches can be used to place sensors at various locations on a body. Moreover, patches may or may not have a connector for making an electrical connection with data logger 810. The sensors may be coupled directly with data logger 810. In some embodiments, feedback device 815 can be separately attached to the user. In other embodiments, feedback device 815 can be part of the data logger.

While stretch sensors are shown on patch 800, any type of sensor can be used including electromyographical sensors, tilt sensors, etc. Moreover, patch 800 can include any type of clothing including a sock, sleeve, compression sleeves, etc.

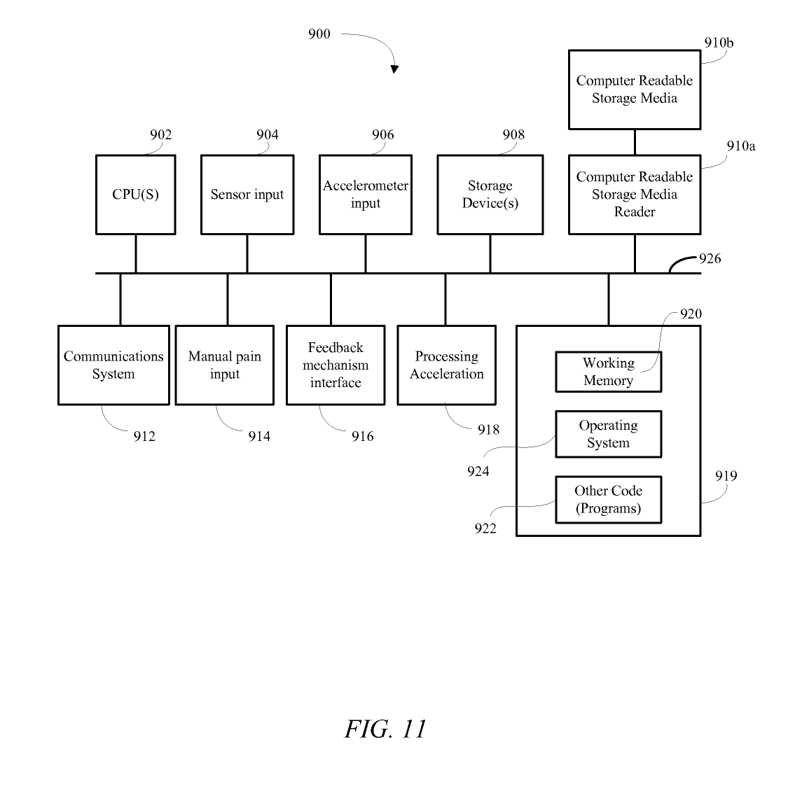

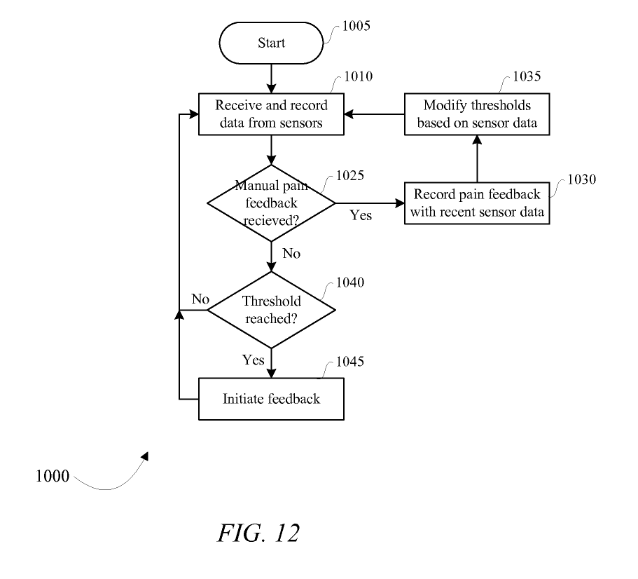

Data logger 810 can be used to record data collected from the various sensors and perform various levels of processing. FIG. 11 shows a block diagram of a computational device 900 that can be used as a data logger. In some embodiments of the invention, computational device 900 can be used to perform the process shown in FIG. 12. The drawing illustrates how individual system elements can be implemented in a separated or more integrated manner. The computer 900 is shown having hardware elements that are electrically coupled via bus 926. The hardware elements can include processor 902, sensor input 904, accelerometer (e.g., tilt) input 906, storage device 908, computer-readable storage media reader 910a, communications system 912, manual pain input 914, zapper interface 916, processing acceleration unit 918, such as a DSP or special-purpose processor, and memory 918. Communications system 912 can communicatively couple the computational device 900 with another computer, for example, using USB, Bluetooth, or WiFi. The computer-readable storage media reader 910a can be further connected to a computer-readable storage medium 910b, the combination comprehensively representing remote, local, fixed, and/or removable storage devices plus storage media for temporarily and/or more permanently containing computer-readable information. In some embodiments, storage device 908, computer-readable storage medium 910b, and memory 919 can all be portions of the same memory.

Computational device 900 can also include software elements, shown as being currently located within working memory 920, including an operating system 924 and other code 922, such as a program designed to implement methods and/or processes described herein. In some embodiments, other code 922 can include software that can be used to store skeletal-muscular position data, determining whether a threshold value(s) has been met, and/or adjusting one or more threshold values in response to input from the sensors and/or the user. In some embodiments a threshold value can include a time, skeletal-muscular position, tilt or a combination thereof. For instance, an extreme stretch in one or more muscles or joints be trigger a threshold value. In other embodiments a lower stretch held for a period of time may also be considered threshold value. It will be apparent to those skilled in the art that substantial variations can be used in accordance with specific requirements. For example, customized hardware might also be used and/or particular elements might be implemented in hardware, software (including portable software, such as applets), or both. Further, connection to other computing devices such as network input/output devices can be employed.

In some embodiments, computational device 900 can receive sensor data, tilt data, and/or manual pain data through the various inputs and store such data within the storage device 908. Data can be collected at various intervals. In some embodiments, computational device 900 can process the data and determine, based on previous input, when a user is nearing a painful situation. In such cases computational device 900 can activate a feedback mechanism through feedback mechanism interface 916.

In some embodiments, a data logger can be a portable battery operated device. The device can be made to fit within a user's pocket or clipped to an article of clothing using a physical clip. In some embodiments, a data logger can include enough memory to log data over periods of tem extend for up to 24 hours, 48 hours, 72 hours, a week, etc. In some embodiments a data logger can include a USB, Bluetooth, or wireless port that can be used to gather data from sensors or download to a computer for analysis.

In some embodiments, when a user encounters a painful situation, the user can so indicate by engaging a feedback mechanism, such as a button on the data logger. When the feedback mechanism is engaged, data can be collected from the sensors to record the position of the user when the painful situation occurred. The data recorded prior to the painful situation and/or during the situation can be used to determine a position threshold that should be avoided. The position threshold can include not only the position of the sensors relative to one another or relative to a fixed point, but also can include the motion of the sensors over time just prior to the painful event. For example, a threshold can also include the amount of time the patient maintained the position, tilt or accelerometer data, a combination of motion and position data, a series of positions or motions, etc.

This data can be used for a number or purposes. First the data can be used for treatment purposes. A physician, physical therapist, or clinician can provide medical treatment based on the knowledge gleaned about the position and/or motion of the person before and during a painful event. The data can also be used in conjunction with a feedback mechanism. The data logger can provide feedback to the user (e.g., using feedback device 815) when the user approaches a position or when their motion is similar to the motion related to the painful event. In some embodiments, the painful event can be replaced with a poor posture configuration that one can use to correct their posture. If a poor posture is maintained for a certain amount of time then the device can be triggered to provide feedback to the user.

FIG. 12 shows a flow chart of process 1000 that can be used by a data logger to record data and/or provide feedback according to one embodiment of the invention. Process 1000 can start at block 1005. At block 1010 data logger can receive and record data from sensors. The data logger can record data from any number of sensors including stretch sensors or tilt sensors, to name a couple. The data can include resistance data that is related to the stretch of a sensor, or the position of a sensor relative to another point on a body. The data can also include voltages that are also related to the stretch of a sensor, or the position of a sensor relative to another point on a body. The data can also be voltage, current, resistance, or other data that is representative of the tilt of a sensor relative to some fixed line, such as vertical. Data can be received continuously or periodically. The data can be time stamped and/or recorded either continuously or periodically as well.

At block 1025 process 1000 can determine whether the user has manually indicated that they are in pain. This information can be indicted by the user engaging a button at the data logger or connected with the data logger. The user may also indicate a painful situation audibly. In response to receiving a manual indication of pain, the feedback and time can be recorded at block 1030. If the data logger records data periodically, the data logger can also record data from the feedback sensors at the same time. In some embodiments, the data logger can modify thresholds that are used to determine when a user is approaching a painful situation at block 1035. Following which, process 1000 can return to block 1010.

If a manual pain indication is not received at block 1025, process 1000 moves on to block 1040. If the data passes a threshold value at block 1040, then feedback is provided to the user at block 1045 and process 1000 returns to block 1010. User feedback can include a vibration the user can feel, a small shock, an audible tone, etc. If the threshold is not reached at block 1040, process 1000 returns to block 1010.

A skeletal-muscular position and motion sensing device has been described in conjunction with a number of embodiments. Various modifications and/or alterations can be made to the embodiments shown and the general description that can apply to any number of configurations.

What is claimed is:

1. A system comprising:
a plurality of stretch sensors configured to be attached to a body, wherein each stretch sensor is also configured to translate a longitudinal stretch into a change in an electrical property of the stretch sensor; and
a data logger coupled with the plurality of stretch sensors and configured to measure the electrical property of the stretch sensors and store electrical property data as a function of time.

2. The system according to claim 1, wherein the electrical property comprises resistance.

3. The system according to claim 1, further comprising a tilt sensor coupled with the data logger, wherein the data logger is configured to record tilt data as a function of time.

4. The system according to claim 3, wherein the tilt sensor comprises an accelerometer or an inclinometer.

5. The system according to claim 1 further comprising a user interface, wherein the data logger is configured to receive and record an indication from the user through the user interface that a painful event has occurred.

6. The system according to claim 5, wherein the user interface comprises a button.

7. The system according to claim 5, wherein the data logger is configured to determine threshold values from the electrical property data received from the stretch sensors and the indication received from the user.

8. The system according to claim 7 further comprising a feedback device coupled with the data logger, wherein the feedback device is configured to provide a user feedback when electrical property data approaches the threshold value.

9. The system according to claim 1 further comprising a patch configured to adhere to a portion of a body, wherein at least one of the plurality of stretch sensors is coupled with the patch and attached to the body using the patch.

10. A system comprising:
a patch configured to couple with a body;
a plurality of sensors coupled with the patch, wherein the plurality of sensors are configured to sense a position of the sensors relative to one another;
memory; and
a controller communicatively coupled with the plurality of sensors and the memory, wherein the controller is configured to receive sensor data and write the sensor data to the memory.

11. The system according to claim 10 further comprising a tilt sensor coupled with the patch and communicatively coupled with the controller, wherein the controller is configured to write tilt data to the memory.

12. The system according to claim 10 further comprising a feedback mechanism communicatively coupled with the controller, wherein the feedback mechanism is configured to provide feedback to a user in response to a signal from the controller, wherein the controller provides a signal to the feedback mechanism when sensor data reaches a threshold value.

13. The system according to claim 12 further comprising a tilt sensor coupled with the patch and communicatively coupled with the controller, wherein the controller provides a signal to the feedback mechanism when one or both of sensor data and tilt data reaches a threshold value.

14. The system according to claim 12 wherein at least one of the sensors comprises a stretch sensor.

15. The system according to claim 12 wherein at least one of the sensors comprises an electromyographical sensor.

16. The system according to claim 12 wherein at least one of the sensors comprises a tilt sensor.

17. A method comprising:
receiving data from a plurality of stretch sensors coupled with a body, wherein the data includes data elements indicating the position of the body;
determining whether one or more data elements approaches a threshold value;
in the event one or more data elements approaches a threshold value, providing feedback to the user.

18. The method according to claim 18 further comprising:
receiving an indication from the user indicating a painful position;
recalculating the threshold value based on the data elements received when the indication was received from the user.

19. The method according to claim 18, wherein the feedback includes either vibration or shock.

20. The method according to claim 18, wherein the data comprises either resistance or voltage.
